(19) United States
(12) Patent Application Publication
(10) Pub. No.: US 2026/0165395 A1
(43) Pub. Date: Jun. 18, 2026

(54) SCENT-BLOCKING MASK

(71) Applicants: Shameka Van Dyke, Waddell, AZ (US); Jeremy Van Dyke, Waddell, AZ (US)

(72) Inventors: Shameka Van Dyke, Waddell, AZ (US); Jeremy Van Dyke, Waddell, AZ (US)

(21) Appl. No.: 19/423,360

(22) Filed: Dec. 17, 2025

Related U.S. Application Data
(60) Provisional application No. 63/734,919, filed on Dec. 17, 2024.

(51) Int. Cl. A41D 13/11; A61L 9/014; A61L 9/04; A61L 9/12; A62B 18/08

(52) U.S. Cl. CPC A41D 13/11; A61L 9/014; A61L 9/048; A61L 9/12; A62B 18/08; A61L 2209/133; A61L 2209/134; A61L 2209/14; A61L 2209/15

(57) ABSTRACT

The invention pertains to a scent-blocking mask designed to cover a user's nose and mouth. The mask comprises a main body with an exterior and interior surface, a carrier attached to the main body, and a scent module that can be inserted into the carrier. The scent module contains a reservoir filled with a scent-emitting substance. An activation mechanism is included to selectively release the scent-emitting substance into the interior layer of the mask. The carrier, which may be made of flexible, polymeric material, has an inner layer that is coupled to the exterior layer of the mask, aligning with an access port to the interior surface. The scent module can be activated to release the scent-emitting substance upon the application of pressure or compression.

### CROSS REFERENCE TO RELATED APPLICATIONS

This application claims benefit from currently pending U.S. Provisional Application No. 63/734,919 titled “Scent-Blocking Mask” and having a filing date of Nov. 17, 2024, all of which is incorporated by reference herein.

### FIELD OF THE INVENTION

The field pertains to personal protective equipment, particularly designed for medical settings, focusing on a mask system and even more specifically a mask that integrates odor-neutralizing features within a protective mask, thereby enhancing comfort and concentration for medical professionals in odor-intensive environments.

### BACKGROUND OF THE INVENTION

The field of medical protective equipment, particularly facial masks, involves devices that provide filtration against harmful particles and pathogens. These masks are essential for healthcare professionals, who routinely operate in environments with exposure to biological hazards. The standard design of medical masks primarily focuses on filtration efficiency to safeguard against airborne contaminants. While this fundamental functionality is critical, the masks do not typically address the issue of odors encountered during medical procedures or patient interactions. These masks typically cover the user's nose and mouth, providing a physical barrier against airborne particles. Some masks may also include additional features, such as exhalation valves, to enhance user comfort and functionality.

Existing medical masks, while effective in protecting against pathogens, fall short in managing odors that may be unpleasant or distracting to healthcare professionals. In certain situations, users may desire to mask unpleasant ambient odors or to enjoy specific scents for personal preference or therapeutic benefits. Traditional masks do not offer a solution to these issues, as they are primarily designed to block particles, not to control the scent within the mask. The persistent odor in clinical settings can hinder performance by decreasing concentration and overall comfort. Current solutions, such as the use of personal odor-neutralizing agents such as toothpaste, are not seamlessly integrated into protective gear, often requiring separate applications that may interfere with the mask's primary function or prove inefficient over extended periods of use.

There is an observable need for medical masks that provide not only the necessary protective filtration but also include integrated mechanisms for odor mitigation. Such advancements would enable medical professionals to maintain focus and professionalism, particularly in environments where unpleasant smells are prevalent. Solutions must ensure that the addition of odor-control features does not compromise the mask's standard protective capabilities, including filtration efficiency and breathability.

What is needed is a facial mask system that incorporates dual functionality—providing effective protective filtration typical of standard medical masks while contemporaneously neutralizing unfavorable odors. The system should facilitate seamless integration of odor-control elements without altering the mask's conventional use, thereby enhancing the user's experience and ensuring sustained comfort and focus, especially during prolonged medical procedures. This would represent an enhancement of traditional protective masks while addressing the shortcomings related to odor interference.

### SUMMARY OF THE INVENTION

The present invention encompasses a scent-blocking mask that can help users manage unpleasant odors in various environments. The mask includes a main body that can be configured to cover a user's nose and mouth, with an exterior layer and an interior layer designed to provide comfort and functionality. The mask can include a carrier that is coupled to the main body. This carrier can be configured as a pocket having a pocket opening, with a carrier inner surface and carrier outer surface. In some embodiments, the carrier inner surface can have an opening that allows scent-emitting substances to diffuse toward the user's breathing area. The carrier inner surface can be coupled to the exterior layer, and the exterior layer can include an access port to facilitate interaction with the interior components.

A scent module can be removably insertable into the carrier, allowing users to customize their experience. The scent module can comprise a reservoir containing a scent-emitting substance and an activation mechanism that can be operated to selectively release the scent-emitting substance onto the interior layer of the mask. The activation mechanism can be configured to release the scent-emitting substance through various methods, such as application of pressure to the scent module, pulling of a release tab, or compression of the scent module. The carrier can be made from flexible, polymeric materials such as polyurethane (PU), thermoplastic polyurethane (TPU), or polyethylene (PE). The scent module can comprise a breakable capsule containing a liquid or gel that encapsulates the scent-emitting substance. In some implementations, the scent-emitting substance can be encapsulated in microcapsules or held within an absorbent matrix positioned within the carrier to regulate the duration and/or intensity of scent release.

The scent-emitting substance can be selected from various options, including essential oils, fragrance oils, infused oils, aromatherapy compounds, or odor-neutralizing agents, allowing users to choose their preferred scent or neutralization approach. The main body can comprise a breathable material configured to facilitate airflow while maintaining containment of the scent-emitting substance. The exterior layer can be treated with oleophobic or hydrophobic coatings to prevent the scent-emitting substance from penetrating through to the exterior surface.

The carrier can be configured with a receiving structure for selectively securing the scent module, allowing users to insert their preferred scent module as needed. The scent module can be configured for removable attachment to the carrier through various mechanisms, such as insertion into a pocket, snap-fit connection, magnetic attachment, hook-and-loop fastening, or clip mechanism. The container holding the scent-emitting substance can be formed from various materials depending on the desired release mechanism. For example, it can be formed from a frangible material configured to rupture upon application of mechanical force, a deformable material with a sealed opening, or a rigid housing with a valve mechanism. The main body can further include features for enhanced comfort and fit, such as adjustable ear loops, a nose wire for conforming to facial contours, or a plurality of pleats for expansion to accommodate different face shapes and sizes.

The invention also encompasses methods of manufacturing the scent-blocking mask. The manufacturing process can include providing a carrier configured with a receiving structure to removably secure a scent module, securing the carrier to an outer layer and securing an inner layer to the outer layer, providing the scent module containing at least one scent-emitting substance, and securing the scent module to the receiving structure. The carrier can be constructed from various materials such as cotton, polyester, non-woven polypropylene, silicone, thermoplastic elastomer (TPE), or breathable polymers, configured to retain the scent module while allowing diffusion of scent toward the user.

The scent module can be designed with various release mechanisms, such as a frangible outer shell made from a biodegradable polymer that ruptures upon application of mechanical pressure, a squeezable container with a valve, or a twist-to-open capsule. The receiving structure can comprise various attachment systems, such as a pocket with a semi-permeable barrier to control diffusion rate, a snap-fit holder, a magnetic mounting system, or an adhesive attachment area. The outer layer can be assembled from multi-layered materials configured to block external odors while maintaining airflow. These multi-layered materials can include activated carbon layers, nanofiber filters, or treated fabric layers that provide both odor blocking and breathability for user comfort.

Aspects and applications of the invention presented here are described below in the drawings and detailed description of the invention. Unless specifically noted, it is intended that the words and phrases in the specification and the claims be given their plain, ordinary, and accustomed meaning to those of ordinary skill in the applicable arts. The inventors are fully aware that they can be their own lexicographers if desired. The inventors expressly elect, as their own lexicographers, to use only the plain and ordinary meaning of terms in the specification and claims unless they clearly state otherwise and then further, expressly set forth the “special” definition of that term and explain how it differs from the plain and ordinary meaning. Absent such clear statements of intent to apply a “special” definition, it is the inventors'intent and desire that the simple, plain and ordinary meaning to the terms be applied to the interpretation of the specification and claims. Aspects and applications of the invention presented here are described below in the drawings and detailed description of the invention.

The inventors are also aware of the normal precepts of English grammar. Thus, if a noun, term, or phrase is intended to be further characterized, specified, or narrowed in some way, then such noun, term, or phrase will expressly include additional adjectives, descriptive terms, or other modifiers in accordance with the normal precepts of English grammar. Absent the use of such adjectives, descriptive terms, or modifiers, it is the intent that such nouns, terms, or phrases be given their plain, and ordinary English meaning to those skilled in the applicable arts as set forth above.

Further, the inventors are fully informed of the standards and application of the special provisions of 35 U.S.C. § 112(f). Thus, the use of the words “function,” “means” or “step” in the Detailed Description or Description of the Drawings or claims is not intended to somehow indicate a desire to invoke the special provisions of 35 U.S.C. § 112(f), to define the invention. To the contrary, if the provisions of 35 U.S.C. § 112(f) are sought to be invoked to define the inventions, the claims will specifically and expressly state the exact phrases “means for” or “step for, and will also recite the word “function” (i.e., will state “means for performing the function of . . . ”), without also reciting in such phrases any structure, material or act in support of the function. Thus, even when the claims recite a “means for performing the function of . . . ” or “step for performing the function of . . . ,” if the claims also recite any structure, material or acts in support of that means or step, or that perform the recited function, then it is the clear intention of the inventors not to invoke the provisions of 35 U.S.C. § 112(f). Moreover, even if the provisions of 35 U.S.C. § 112(f) are invoked to define the claimed inventions, it is intended that the inventions not be limited only to the specific structure, material or acts that are described in the preferred embodiments, but in addition, include any and all structures, materials or acts that perform the claimed function as described in alternative embodiments or forms of the invention, or that are well known present or later-developed, equivalent structures, material or acts for performing the claimed function.

### BRIEF DESCRIPTION OF DRAWINGS

A more complete understanding of the present invention may be derived by referring to the detailed description when considered in connection with the following illustrative figures. In the figures, like reference numbers refer to like elements or acts throughout the figures.

FIG. 1a shows an isometric view and close-up view of the scent-blocking mask in accordance to one or more embodiments;

FIG. 1b shows a closeup view of FIG. 1a of the scent blocking mask in accordance to one or more embodiments;

FIG. 2 shows a back close-up view of the scent blocking mask in accordance to one or more embodiments; and

FIG. 3 shows a back view of the scent blocking mask in accordance to one or more embodiments; and

FIG. 4 shows an isometric view of another embodiment of the scent blocking mask in accordance to one or more embodiments.

Elements and acts in the figures are illustrated for simplicity and have not necessarily been rendered according to any particular sequence or embodiment.

### DETAILED DESCRIPTION OF THE INVENTION

In the following description, and for the purposes of explanation, numerous specific details are set forth to provide a thorough understanding of the various aspects of the invention. It will be understood, however, by those skilled in the relevant arts, that the present invention may be practiced without these specific details. In other instances, known structures and devices are shown or discussed more generally to avoid obscuring the invention. In many cases, a description of the operation is sufficient to enable one to implement the various forms of the invention, particularly when the operation is to be implemented in software. It should be noted that there are many different and alternative configurations, devices, and technologies to which the disclosed inventions may be applied. The full scope of the inventions is not limited to the examples that are described below.

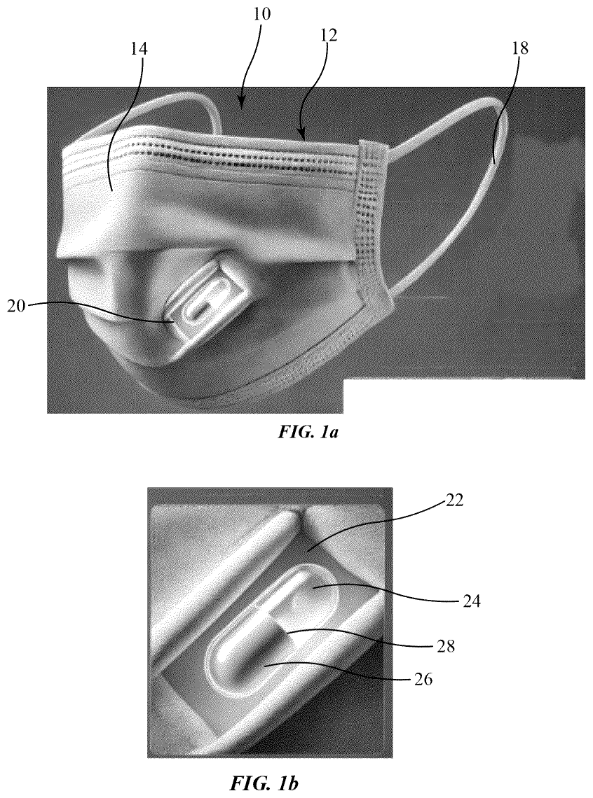

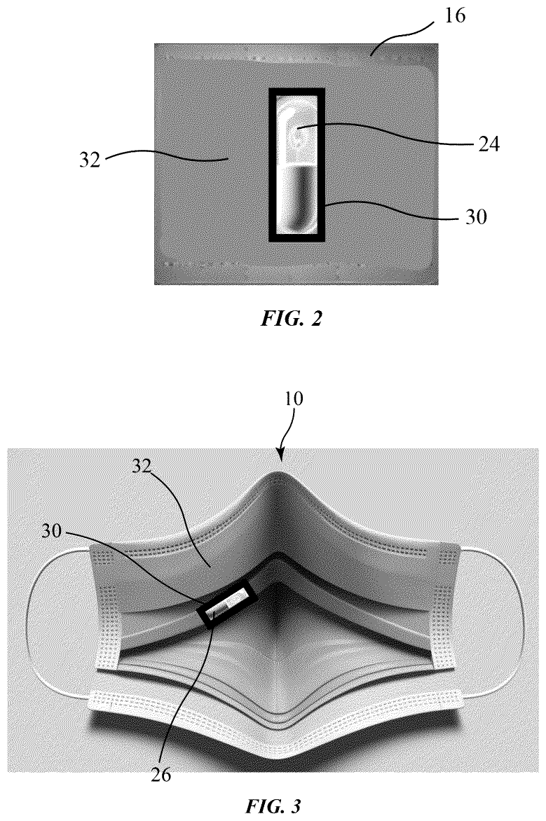

Referring initially to FIGS. 1-3 , a scent-blocking mask is shown generally at 10. The scent-blocking mask 10 can comprise a main body 12 having an exterior layer 14 and an interior layer 16, wherein the main body 12 is configured to cover a user's nose and mouth during use. The exterior layer 14 is the outermost layer of the mask, which is exposed to the external environment and can serve as the first barrier against airborne contaminants and odors, while the interior layer 16 is the innermost layer, which is positioned in direct contact with or in close proximity to the user's face. This dual-layer construction provides a balance between external odor blocking and internal scent delivery that has not been achieved in prior mask designs.

The exterior layer 14 can be made from a material that can repel water, bodily fluids, and external contaminants, wherein the material can be such as, for example, spunbond polypropylene, melt-blown polypropylene, spunlace nonwoven, polyester, polyethylene nonwoven, or the like. The exterior layer 14 can be treated with at least one oleophobic coating or hydrophobic coating to prevent the scent-emitting substance from penetrating through to an exterior surface of the exterior layer 14. The oleophobic coating can be applied through such as, for example, plasma treatment, chemical vapor deposition, spray coating, dip coating processes, or the like and can comprise such as, for example, fluoropolymer-based compounds, silicone-based treatments, nano-particle coatings or the like that can create a molecular barrier with contact angles greater than 110 degrees for oils and greater than 150 degrees for water.

The exterior layer 14 can be coupled to the interior layer 16, wherein the interior layer 16 can be made from the same or similar material as the exterior layer 14, or the interior layer 16 can be a hydrophilic material that can absorb a scent-emitting substance and/or moisture from the wearer's breath. The interior layer 16 can be configured to be comfortable for the wearer and can be made from materials such as, for example, cotton, viscose, polyester, lyocell, spunlace nonwoven fabric, or the like. In certain embodiments where the interior layer 16 comprises a hydrophilic material, the layer can be treated to enhance scent absorption and gradual release characteristics, providing a reservoir effect that maintains scent presence over extended wearing periods. The interior layer 16 can have a basis weight ranging from such as, for example, approximately 20 gsm to 100 gsm (grams per square meter) to balance absorbency with breathability.

In embodiments, the combination of exterior layer 14 materials and the interior layer 16 materials can allow the mask 10 to maintain its protective odor-blocking function while maximizing the wearer's comfort and enabling controlled scent delivery to the breathing zone. The exterior layer 14 and the interior layer 16 can be coupled together by such as, for example, ultrasonic welding, heat sealing, adhesive bonding, stitching or sewing, lamination, or the like. The coupling method can be selected based on the materials used and the desired flexibility and durability characteristics of the finished mask. Ultrasonic welding can be particularly advantageous as it creates bonded seams without introducing additional materials that might interfere with scent delivery or cause allergic reactions.

In other embodiments, a middle layer can be positioned between the exterior layer 14 and the interior layer 16 and can act as a protective filtration layer that can block external odors while allowing controlled passage of internally released scents. This middle layer can comprise activated such as, for example, carbon material, zeolite particles, nanofiber filters or the like with fiber diameters less than 500 nanometers or treated fabric layers impregnated with odor-neutralizing compounds. The middle layer can block external odors (which requires dense filtration) while maintaining breathability (which requires adequate airflow). In certain embodiments, incorporating activated carbon, the carbon can be in granular form with particle sizes ranging from such as, for example, 0.5 mm to 4.0 mm, or can be incorporated into a non-woven fabric structure through adhesive bonding or thermal bonding processes. The activated carbon can provide surface areas such as, for example, exceeding 500-1000 m²/g, enabling adsorption of volatile organic compounds and malodorous molecules while allowing oxygen and carbon dioxide to pass through for comfortable breathing.

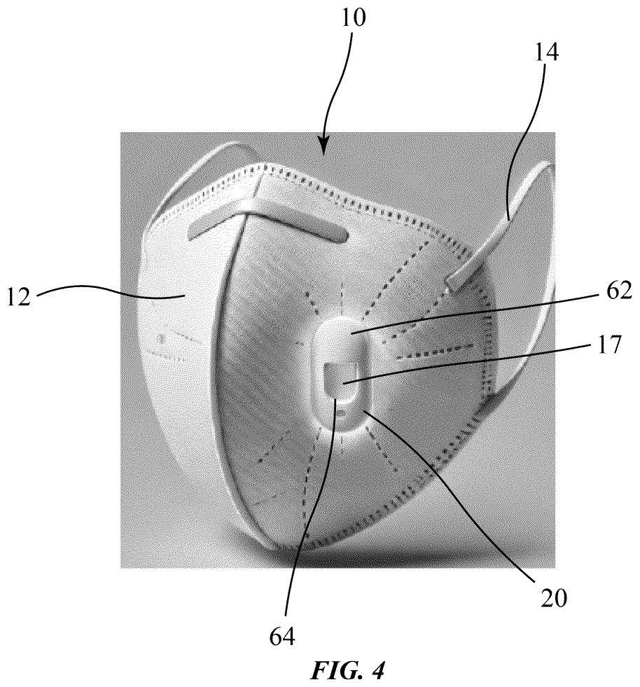

In embodiments, the scent blocking mask 10 can further comprise a carrier 20 wherein the carrier 20 can comprise a pocket 26, a carrier inner surface 32, and a carrier outer surface 22, wherein the carrier inner surface and/or carrier outer surface can be coupled to the main body 12 by either the exterior layer 14 and/or the interior layer 16. This dual-surface coupling configuration provides structural stability while enabling precise positioning of the scent source relative to the user's breathing zone allowing the scent sources too not be too far from the nose and mouth or too close. The carrier inner surface can be coupled to the exterior layer wherein the exterior layer can have an access port 17, as shown in FIG. 4, or it can be positioned on or in contact with the interior layer wherein the access port can comprise a fluid communication channel between the carrier and the interior layer configured to deliver the scent-emitting substance while maintaining the structural and filtration integrity of the main body.

The coupling between the carrier 20 and the main body 12 can be done by such as, for example, ultrasonic welding, heat sealing, adhesive bonding, stitching or sewing, lamination, or the like. The carrier 20 can be positioned on the main body 12 corresponding to the anatomical position of the user's philtrum (the area between the nose and upper lip approximately 10 mm to 50 mm below the nose bridge), where scent receptors are most sensitive and where airflow during inhalation is concentrated. The positioning of the carrier 20 can be achieved by coupling the carrier 20 to the interior layer 16 at a location such as, for example, approximately 10 mm to 50 mm below the expected position of the nose bridge when the mask 10 is worn, optimizing scent delivery efficiency while maintaining user comfort.

In embodiments, the carrier 20 can have a pocket opening 28 which can allow for the insertion and removal of a scent module 24, providing users with the flexibility to customize their scent experience based on changing environmental conditions, personal preferences, or the depletion of scent material. In certain embodiments, the pocket opening 28 can be omitted, and the scent module 24 can be permanently coupled inside of the carrier 20, providing a simplified construction suitable for single-use or disposable mask applications.

In embodiments, the carrier 20 can be made from sew-in plastic or can comprise, for example, a flexible, polymeric material, which can be selected from at least one of polyurethane (PU), thermoplastic polyurethane (TPU), or polyethylene (PE) configured to securely contain and distribute the scent-emitting substance while allowing the controlled release of a scent-emitting substance through micro-perforations or a semi-permeable structure. The carrier 20 materials can include flexibility allowing the carrier to conform to facial contours without creating pressure points, durability which can maintain structural integrity through repeated compression cycles during breathing, and resistance to various environmental factors including oils and fragrances that could otherwise degrade conventional mask materials.

In embodiments, the carrier 20 can have a carrier opening 30 on the carrier inner surface 32 that incorporates a diffusion control structure. The carrier opening 30 can include such as, for example, a mesh, micro-perforations, semi-permeable structure or the like which can allow the scent-emitting substance to diffuse in a controlled manner to saturate the interior layer 16. The micro-perforation or semi-permeable structure can comprise materials such as, for example, wool felt, bamboo fiber, cellulose acetate, silicone carriers or the like with controlled porosity, laminated composites, synthetic mesh materials, or the like. In certain embodiments, micro-perforations can have diameters ranging from such as, for example, approximately 50 micrometers to 500 micrometers and density of 10 to 400 per square centimeter, with a perforation density ranging from such as, for example, 10 to 400 perforations per square centimeter. The specific perforation sizing can provide a balance wherein the perforations are large enough to permit adequate scent molecule diffusion wherein the fragrance molecules can have molecular weights between 150-300 daltons and effective diameters less than 1 nanometer, and can be small enough to prevent bulk flow of liquid scent material that could saturate the interior layer 16 too rapidly or leak onto the user's skin.

In embodiments, the carrier opening 30 with its diffusion control structure can contain the scent module 24 and provide a barrier between the user and scent-emitting substance, preventing direct skin contact with potentially irritating concentrated fragrance compounds while still enabling effective scent delivery to the breathing zone. The barrier can keep the concentrated fragrance compound from the user's skin. In other embodiments, where the scent module 24 can gradually release or where the scent-emitting substance is formulated in a non-irritating form, the carrier opening 30 can be omitted from the carrier 20, simplifying the construction while maintaining scent delivery functionality. In certain embodiments, the carrier 20 can be formed around the scent module 24 during manufacturing, creating an integrated assembly, or alternatively the pocket 26 can be configured with an opening that allows the user to selectively choose the scent module 24 and place it within the pocket 26 at the time of use.

Referring to FIG. 4, in other embodiments, the carrier 20 can comprise a rigid or semi-rigid plastic shell that provides an alternative structural approach to scent module containment and protection. This hard plastic shell configuration can be formed from materials such as polycarbonate, acrylonitrile butadiene styrene (ABS), polypropylene, polyethylene terephthalate (PET), or the like selected for their dimensional stability, impact resistance, and impermeability to scent compounds. The hard-shell carrier 20 can have a wall thickness ranging from such as, for example, 0.5 mm to 5.0 mm, providing structural rigidity to protect the scent module 24 from inadvertent crushing or deformation during storage or transport.

The hard-shell carrier 20 can accept the scent module 24 either removably or permanently through an outer opening 64 that can be configured such as, for example, a snap-fit closure, threaded connection, sliding panel, hinged lid or the like. In removable configurations, the outer opening 64 can incorporate retention features such as, for example, detents, snap tabs, bayonet-style locking mechanisms or the like that can provide tactile feedback when the scent module 24 is properly seated, ensuring reliable containment during use. The hard-shell design provides particular advantages in applications where the scent module 24 must be activated through mechanical pressure or compression, as the rigid structure can transmit applied force efficiently to the scent module 24 without flexing or deforming.

The carrier 20 in its hard-shell configuration can be integrated with the exterior layer 14 through various attachment methods including such as, for example, adhesive bonding, snap-fit attachment to pre-formed receptacles in the main body 12, or through-bolting with low-profile fasteners or the like. An inner opening in the hard-shell carrier 20 can be positioned to align with a corresponding aperture in the exterior layer 14, creating a controlled pathway that allows the scent-emitting substance to saturate the interior layer 16 while preventing the substance from escaping to the external environment. This inner opening can incorporate the same types of diffusion control structures described previously such as, for example, micro-perforations, semi-permeable membranes, mesh materials or the like to regulate scent release rate and prevent over-saturation of the interior layer 16.

In certain embodiments, the hard-shell carrier 20 can incorporate a valve mechanism that provides user-adjustable control over scent release. The valve mechanism can comprise a rotating disk with apertures of varying sizes, a sliding shutter, an iris-style diaphragm or the like that can be manipulated by the user to increase or decrease the effective opening size of the inner opening. The adjustable release mechanism provides users with real-time control over scent intensity based on changing environmental conditions or personal preference, without requiring removal or replacement of the scent module 24. For example, a user entering an environment with particularly strong odors could increase the valve opening to enhance scent delivery, then reduce the opening when moving to a less challenging environment to conserve scent material.

Referring back to FIGS. 1-3 , the scent module 24 comprises a reservoir configured to contain the scent-emitting substance in a stable, controlled form until user activation is desired. The scent emitting substance contained within the reservoir can be selected from a wide range of compounds formulated to provide specific sensory, therapeutic, or odor-neutralizing effects. In embodiments, the scent-emitting substance can comprise essential oils such as, for example, lavender (Lavandula angustifolia), peppermint (Mentha piperita), chamomile (Matricaria chamomilla), frankincense (Boswellia serrata), geranium (Pelargonium graveolens), citrus oils including lemon (Citrus limon), orange (Citrus sinensis), bergamot (Citrus bergamia), grapefruit (Citrus paradisi), fragrance oils, aromatherapy compounds, or the like. Each of these scent-emitting substances can be selected based on specific functional properties lavender for its calming and stress-reducing effects which can be particularly valuable for healthcare workers in high-stress environments, peppermint for its invigorating properties and ability to promote alertness, chamomile for its soothing characteristics, frankincense for its grounding and anxiety-reducing properties, and citrus oils for their uplifting and energizing effects.

The scent-emitting substance can provide a subtle, comforting fragrance while enhancing the user's experience in environments characterized by unpleasant ambient odors. These challenging environments can include such as, for example, patient rooms (where bodily fluids, medications, and illness-related odors may be present), operating rooms (with odors from cauterization, disinfectants, and biological tissues), cadaver labs and anatomy laboratories (with preservation chemical odors), wound care and burn units (where wound drainage, necrotic tissue, and topical medications create complex malodor profiles), emergency rooms (with diverse trauma-related odors), infectious disease units, or the like.

In certain embodiments, the scent-emitting substance can comprise odor-neutralizing agents rather than or in addition to pleasant fragrances. These odor-neutralizing agents can include compounds such as, for example, cyclodextrin complexes that physically encapsulate malodor molecules, activated charcoal in colloidal suspension, zinc ricinoleate which can be chemically bound to odor molecules, essential oils with known antimicrobial properties (such as tea tree oil, eucalyptus, or thyme oil) that can reduce odor-causing bacterial growth, or enzymatic compounds that break down odor-causing organic molecules.

In embodiments, the scent module 24 can be activated by an activation mechanism operable to selectively release the scent-emitting substance into the interior layer 16, providing users with control over the timing and intensity of scent delivery. The activation mechanism can be configured to release the scent-emitting substance upon the application of such as, for example, pressure, compression, piercing, crushing, twisting, pulling, or the like to the scent module 24, enabling single-handed operation while the mask 10 is worn on the user's face.

The activation mechanism prevents premature leakage during storage, transport, and initial mask donning, and enables targeted scent delivery precisely when the user needs it. This controlled release addresses a significant limitation of prior art scented masks: the inability to conserve scent material until actually needed. In a typical healthcare shift, a user might wear a mask for 8-12 hours but only encounter strong malodors during specific procedures or in specific locations. The scent activated mask allows the user to activate scent release only during these challenging periods, potentially extending the useful life of a single scent module 24 across multiple work shifts rather than requiring replacement after each use.

In embodiments, the scent module 24 can be configured as such as, for example, hard gelatin capsules, microcapsules, frangible plastic capsules, coated blisters, encapsulated gels, pressurized capsules, crushable glass ampules, squeezable polymer pouches, twist-to-open containers, or the like, which can allow the user to easily activate the scent release upon crushing, piercing, or mechanical manipulation wherein the mechanical force to crush and release the mechanism can be such as, for example, approximately from 2 Newtons to 30 Newtons. In other embodiments, the scent module 24 can have a multi-chamber design with multiple separate reservoirs containing different scent-emitting substances that can be released sequentially or simultaneously. For example, a two-chamber scent module 24 could contain a strong initial burst scent in a first chamber (for immediate odor masking) and a gentler, longer-lasting scent in a second chamber (for sustained comfort over extended periods). The multi-chamber design can be activated through progressive compression (first chamber releases at lower force, second chamber at higher force) or through separate activation mechanisms. The sequential release capability represents a significant advancement over single-payload systems, providing users with adaptable scent delivery that can respond to changing environmental challenges over the course of a work shift.

In certain embodiments, the physical dimensions of the scent module 24 can be selected to balance several competing requirements: sufficient volume to contain an effective dose of scent-emitting substance which can be such as, for example, 0.1 to 2.0 milliliters of liquid scent material, compact size to avoid creating uncomfortable pressure points or bulk on the mask 10, and appropriate geometry to facilitate reliable activation through the carrier 20. In typical embodiments, the scent module 24 can have a largest dimension ranging from such as, for example, 5 mm to 100 mm, a thickness ranging from 1 mm to 30 mm, and an internal volume ranging from such as, for example, 0.05 to 6.0 cubic centimeters. These dimensional specifications ensure that the scent module 24 can be comfortably positioned within the carrier 20 without creating noticeable protrusions that would interfere with mask fit or user comfort.

A method of manufacturing a scent-blocking mask 10 can comprise providing a carrier 20 with a pocket 26 having a scent module 24 containing at least one scent-emitting substance. The carrier 20 can be pre-formed through various manufacturing techniques wherein the thermoplastic materials can be such as, for example, polyurethane (PU), thermoplastic polyurethane (TPU), polyethylene (PE), or the like the carrier 20 can be formed through injection molding, vacuum forming, or blow molding processes. In injection molding embodiments, the carrier 20 can be formed in a multi-cavity mold at temperatures ranging from 130° C. to 260° C. (depending on the specific polymer selected), with injection pressures ranging from 30 to 200 MPa and cycle times ranging from 15 to 60 seconds per part. The mold can be designed to create the pocket 26 as an integral feature of the carrier 20, with pocket dimensions precisely controlled to accept the scent module 24 with a clearance fit of approximately 0.5 mm to 5.0 mm, ensuring secure retention while allowing easy insertion during assembly.

The method can further comprise assembling an outer layer 14 (exterior layer) configured to be coupled to the carrier 20. The outer layer 14 can be prepared through various textile manufacturing or film extrusion processes depending on the selected material. In embodiments utilizing non-woven materials such as spunbond polypropylene or melt-blown polypropylene, the outer layer 14 can be manufactured through continuous web processes at line speeds ranging from 100 to 600 meters per minute, with basis weights controlled to range from 15 to 50 grams per square meter (gsm). The outer layer 14 can be treated with oleophobic or hydrophobic coatings through spray application, dip coating, or plasma treatment processes, with the coating applied at coverage rates ranging from 0.5 to 5.0 grams per square meter to achieve the desired liquid repellency characteristics.

The method can comprise assembling an inner layer 16 to be coupled to the outer layer 14, creating a multi-layer structure that provides both comfort and functionality. The inner layer 16 can be prepared from materials selected for their softness, breathability, and scent-absorbing properties. In embodiments, the inner layer 16 can be formed from such as, for example, cotton, viscose, lyocell, spunlace nonwoven fabrics or the like having basis weights ranging from 30 to 80 gsm. The inner layer 16 can undergo pre-treatment processes to enhance its scent-absorbing properties, such as treatment with hydrophilic surfactants, application of superabsorbent polymer coatings, or incorporation of activated carbon particles at loading levels ranging from 5% to 20% by weight.

The carrier 20 can be constructed from a material selected from the group consisting of such as, for example, cotton, polyester, non-woven polypropylene, silicone, thermoplastic elastomer (TPE), a breathable polymer, or the like configured to retain the scent module 24 securely while allowing diffusion of the scent-emitting substance. The material can be tested for vapor transmission rate (measured in grams per square meter per 24 hours) to ensure that it provides adequate diffusion of volatile scent compounds (target transmission rates ranging from 500 to 5000 g/m²/24 hr) while preventing liquid penetration.

The pocket 26 within the carrier 20 can have a semi-permeable barrier to control the rate of diffusion of the activated scent-emitting substance that can extend scent duration and prevents over-saturation of the interior layer 16. The semi-permeable barrier can comprise materials such as microporous membranes with pore sizes ranging such as, for example, from 0.1 to 30 micrometers, hydrophilic polymers with controlled water content, or laminated structures combining permeable and impermeable layers. In manufacturing embodiments, the semi-permeable barrier can be integrated into the carrier 20 during the forming process or can be separately manufactured and subsequently attached to the carrier inner surface 32 through adhesive bonding, thermal welding, or ultrasonic bonding processes.

In certain embodiments, the outer layer 14 can be assembled from a multi-layered material configured to block external odors while maintaining airflow sufficient for comfortable breathing. This multi-layered structure can comprise multiple distinct layers, each contributing specific functional properties. In embodiments, a three-layer structure can comprise such as, for example, an outer protective layer of spunbond polypropylene (15-25 gsm) providing mechanical protection and water repellency, a middle filtration layer of melt-blown polypropylene (20-40 gsm) providing fine particle filtration and odor adsorption, and an inner comfort layer of spunlace nonwoven (30-50 gsm) providing softness and moisture management. These layers can be bonded together through thermal point bonding, where heated pins or rollers create fusion points at temperatures ranging from such as, for example, 120° C. to 200° C., with bonding patterns covering 10% to 60% of the surface area to maintain breathability while providing secure inter-layer attachment.

The inner layer 16 can be constructed from an absorbent material that captures the released scent-emitting substance to facilitate gradual diffusion within the mask 10 over extended periods of use. The absorbent material can be selected based on its specific absorption capacity for the scent-emitting substances being used. In embodiments, the inner layer 16 can comprise cellulosic fibers (cotton, viscose, lyocell) having moisture regain values ranging from 8% to 15% at standard atmospheric conditions (21° C., 65% relative humidity), providing absorption of aqueous and alcoholic scent formulations. The inner layer 16 can be treated with surface-active agents or humectants (such as glycerin, propylene glycol, or polyethylene glycol) at application rates ranging from 1% to 10% by weight to enhance absorption kinetics and distribution of the scent-emitting substance throughout the layer.

The method can comprise an activating step wherein the activation step can comprise providing a mechanical crushing element integrated into the carrier 20 or allowing the user to manually crush the scent module, enabling the user to apply sufficient force to crush the scent module 24 and release the scent-emitting substance. In certain manufacturing embodiments, the carrier 20 can have such as, for example raised ridges, embossed patterns, localized thinned regions or the like positioned adjacent to where the scent module 24 will be located, facilitating more efficient force transmission from the user's fingers through the carrier material to the scent module 24. These force concentration features can reduce the required activation force by such as, for example, 20% to 70% compared to carriers without such features, improving ease of use particularly for users wearing thick protective gloves. The scent-emitting substance can comprise a liquid or gel fragrance infused with essential oils, synthetic aromas, or functional scent agents, such as aromatherapy compounds, formulated specifically for the enclosed environment within a face mask. The scent formulations can be prepared through mixing processes that ensure homogeneous distribution of active scent compounds in the carrier vehicle.

In embodiments, the carrier 20 can be attached to the outer layer 14 using heat sealing, ultrasonic welding, or adhesive bonding to create a secure and airtight connection that prevents scent leakage to the external environment. The scent module 24 can include a pressure-sensitive activation mechanism, enabling controlled release of the scent-emitting substance upon user interaction at a precisely determined force threshold. In manufacturing embodiments, the pressure-sensitive activation mechanism can be calibrated through selection of specific shell materials, wall thicknesses, and geometric configurations.

The method can further comprise incorporating additional functional features into the mask 10 assembly to enhance performance, comfort, or user convenience. These additional features can include adjustable ear loops manufactured from elastic materials (such as knitted elastic bands, braided elastomers, or extruded thermoplastic elastomer straps) with lengths adjustable through slider mechanisms or tied knots, enabling users to achieve proper fit across varying head sizes. The ear loops can be attached to the main body 12 through sewing, ultrasonic welding, adhesive bonding, or mechanical fastening at attachment points positioned to distribute tensile loads evenly across the mask edges.

In conclusion, the present invention provides a scent-blocking mask that combines functionality and comfort. The mask provides a barrier between the user and the environment, while also delivering a pleasant scent experience. The mask's design allows for the scent module to be easily inserted and removed, and for the scent-emitting substance to be released in a controlled manner. The mask's materials and coatings ensure durability and effectiveness, making the mask suitable for a variety of uses.

It is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only and is not intended to limit the scope of the present disclosure, which is defined solely by the claims. Accordingly, embodiments of the present disclosure are not limited to those precisely as shown and described.

Certain embodiments are described herein, including the best mode known to the inventors for carrying out the methods and devices described herein. Of course, variations on these described embodiments will become apparent to those of ordinary skill in the art upon reading the foregoing description. Accordingly, this disclosure includes all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

We claim:

1. A scent-blocking mask, comprising:
a main body having an exterior layer and an interior layer wherein the main body is configured to cover a user's nose and mouth;
a carrier coupled to the main body; and
a scent module removably insertable into the carrier wherein the scent module comprises a reservoir containing a scent-emitting substance and an activation mechanism operable to selectively release the scent-emitting substance onto the interior layer of the mask.

2. The scent blocking mask of claim 1, wherein the carrier comprises a pocket having a pocket opening, a carrier inner surface and carrier outer surface.

3. The scent blocking mask of claim 2, wherein the carrier inner surface has a carrier opening for allowing the scent emitting substance to diffuse toward the carrier inner surface.

4. The scent blocking mask of claim 2, wherein the carrier inner surface is coupled to the exterior layer wherein the exterior layer has an access port wherein the access port comprises a fluid communication channel between the carrier and the interior layer configured to deliver the scent-emitting substance while maintaining the structural and filtration integrity of the main body.

5. The scent blocking mask of claim 1, wherein the activation mechanism is configured to release the scent-emitting substance upon at least one of application of pressure to the scent module, pulling of a release tab, or compression to the scent module.

6. The scent blocking mask of claim 2, wherein the carrier is selected from at least one of polyurethane (PU), thermoplastic polyurethane (TPU), polyethylene (PE) or medical grade elastomeric combinations thereof.

7. The scent blocking mask of claim 3, wherein the scent module comprises a breakable capsule containing a liquid or gel that encapsulates the scent emitting substance.

8. The scent blocking mask of claim 5, wherein the scent-emitting substance is encapsulated in microcapsules or held within an absorbent matrix positioned within the carrier to regulate at least one of the duration or intensity of scent release.

9. The scent blocking mask of claim 8, wherein the scent emitting substance is an essential oil, fragrance oils, infused oils, aromatherapy compound, or odor neutralizing agent.

10. The scent blocking mask of claim 1, wherein the main body comprises a breathable material configured to facilitate airflow while maintaining containment of the scent-emitting substance.

11. The scent blocking mask of claim 1, wherein the exterior layer is treated with at least one oleophobic coating or hydrophobic coating to prevent the scent emitting substance from penetrating through to an exterior surface of the exterior layer.

12. The scent blocking mask of claim 1, wherein the carrier is configured with a receiving structure for selectively securing the scent module, allowing a user to insert their preferred scent module.

13. The scent blocking mask of claim 1, wherein the scent module is configured for removable attachment to the carrier through at least one of insertion into a pocket, snap-fit connection, magnetic attachment, hook-and-loop fastening, or clip mechanism.

14. The scent blocking mask of claim 1, wherein the container is formed from at least one of a frangible material configured to rupture upon application of mechanical force, a deformable material with a sealed opening, or a rigid housing with a valve mechanism.

15. The scent-blocking mask of claim 1, wherein the main body further comprises at least one of adjustable ear loops, a nose wire for conforming to facial contours, or a plurality of pleats for expansion.

16. A method of manufacturing a scent-blocking mask, comprising the acts of:
providing a carrier configured with a receiving structure to removably secure a scent module;
securing the carrier to an outer layer and securing an inner layer to the outer layer;
providing at least one of the scent module containing at least one scent-emitting substance; and
securing the scent module to the receiving structure.

17. The method of claim 16, wherein the carrier is constructed from a material selected from at least one of cotton, polyester, non-woven polypropylene, silicone, thermoplastic elastomer (TPE), or a breathable polymer, configured to retain the scent module while allowing diffusion of scent.

18. The method of claim 16, wherein the scent module comprises at least one of a frangible outer shell made from a biodegradable polymer that ruptures upon application of mechanical pressure, a squeezable container with a valve, or a twist-to-open capsule.

19. The method of claim 16, wherein the receiving structure comprises at least one of a pocket with a semi-permeable barrier to control diffusion rate, a snap-fit holder, a magnetic mounting system, or an adhesive attachment area.

20. The method of claim 16, wherein the outer layer is assembled from a multi-layered material configured to block external odors while maintaining airflow, wherein the multi-layered material comprises at least one of activated carbon layers, nanofiber filters, or treated fabric layers.
